(19) United States
(12) Patent Application Publication
(10) Pub. No.: US 2026/0364348 A1
(43) Pub. Date: Nov. 19, 2026

(54) CHASTITY DEVICE

(71) Applicant: RuvoTech LLC, Indianapolis, IN (US)

(72) Inventor: Timothy Munger, Indianapolis, IN (US)

(21) Appl. No.: 19/662,052

(22) Filed: Apr. 29, 2026

Related U.S. Application Data
(63) Continuation-in-part of application No. 19/206,325, filed on May 13, 2025.
(60) Provisional application No. 63/981,989, filed on Feb. 13, 2026.

(51) Int. Cl. A61N 1/36; A61B 5/00; A61F 6/02; A61N 1/32

(52) U.S. Cl. CPC A61N 1/36031; A61B 5/4393; A61F 6/02; A61N 1/321; A61B 2562/0223

(57) ABSTRACT

The present disclosure relates to a chastity device. The chastity device includes a housing, a securing member, at least one magnetic member, one or more sensors, and control circuitry. The securing member is operatively coupled to the housing while being secured to genitalia of a user. Further, the at least one magnetic member is coupled to a contacting member and disposed in the housing. The one or more sensors are configured to detect a magnetic flux due to a displacement of the contacting member. The control circuitry is communicably coupled to the one or more sensors. The control circuitry determines a set of parameters indicative of one or more events associated with the genitalia of the user and the chastity device based at least on the detected magnetic flux. The events include at least a sexual arousal of the user and a tampering condition associated with the chastity device.

### TECHNICAL FIELD

The present disclosure generally relates to a chastity device and more particularly relates to a chastity device, a system for controlling the chastity device, and a method thereof.

### BACKGROUND

Chastity devices have been utilized for centuries for various purposes, including but not limited to personal discipline, behavior modification, and in certain cases, as a tool for sexual control or enhancement within consensual relationships. These devices typically consist of a mechanical lock or restraint that prevents the user from achieving sexual intercourse or arousal.

Traditional male chastity devices are typically made from materials such as plastic, silicone, or metal and are designed to enclose the genitalia in a manner that prevents direct contact or stimulation. The most common form involves a cage-like structure with a locking mechanism that can be secured around the user’s genital area. These devices are typically controlled by the wearer or a third party through a key or other mechanical locking systems.

Further, existing male chastity devices available in the market predominantly consist of rigid cages or belts designed to physically restrict access to the genitals. Many existing male chastity devices are made from hard materials such as metal or plastic, forming a cage around the penis. The rigid construction can cause significant discomfort during long-term wear, especially during normal physiological changes like nocturnal erections. The lack of flexibility can lead to skin irritation, bruising, and other injuries. These devices, while effective in preventing sexual activity, suffer from several significant limitations that can make them uncomfortable, unsafe, and impractical for long-term wear. Most male chastity devices do not incorporate any form of biometric monitoring. As a result, these devices are unable to provide real-time feedback about the user’s physiological state, such as sexual arousal or attempts to tamper with the device. This lack of monitoring makes it difficult to enforce the intended purpose of the device effectively, especially in situations where remote supervision is desired. Current male chastity devices entirely on physical locks and keys for control, without any automated or remote monitoring capabilities. This restricts the ability of a keyholder (the person controlling the device) to monitor and enforce the use of the device in real-time, particularly when the wearer is not physically present.

Therefore, there is a need for a male chastity device that incorporates monitoring capabilities, enhanced safety features, and greater control capabilities, providing a more effective, comfortable, and medically safe solution for long-term wear. Additionally, a method of monitoring and controlling the chastity device in a manner that does not suffer from the aforementioned deficiencies.

### SUMMARY

Various embodiments of the present disclosure disclose a chastity device.

In an embodiment, a chastity device is disclosed. The chastity device includes a housing, a securing member, at least one magnetic member, one or more sensors, and control circuitry. The securing member is operatively coupled to the housing while being secured to genitalia of a user. Further, the at least one magnetic member is coupled to a contacting member and disposed in the housing. The one or more sensors are configured to detect a magnetic flux due to a displacement of the contacting member. Furthermore, the control circuitry is disposed in the housing. The control circuitry is communicably coupled to the one or more sensors. The control circuitry is configured to determine a set of parameters indicative of one or more events associated with the genitalia of the user and the chastity device based at least on the detected magnetic flux. The one or more events include at least a sexual arousal of the user and a tampering condition associated with the chastity device.

In another embodiment, a chastity device is disclosed. The chastity device includes a housing, a securing member, at least one magnetic member, one or more sensors, and control circuitry. The securing member is operatively coupled to the housing while being secured to genitalia of a user. Further, the at least one magnetic member is coupled to a contacting member and disposed in the housing. The one or more sensors are configured to detect a magnetic flux due to a displacement of the contacting member. The chastity device includes an enclosure disposed between the at least one magnetic member and the one or more sensors. Furthermore, the control circuitry is disposed in the housing. The control circuitry is communicably coupled to the one or more sensors. The control circuitry is configured to determine a set of parameters indicative of one or more events associated with the genitalia of the user and the chastity device. The one or more events include at least a sexual arousal of the user and a tampering condition associated with the chastity device.

In yet another embodiment, a method performed by control circuitry is disclosed. The method includes detecting a magnetic flux due to a displacement of the contacting member. Further, the method includes determining a set of parameters indicative of one or more events associated with the genitalia of the user and the chastity device based at least on the detected magnetic flux. The one or more events includes at least a sexual arousal of the user and a tampering condition associated with the chastity device

### BRIEF DESCRIPTION OF THE FIGURES

The following detailed description of illustrative embodiments is better understood when read in conjunction with the appended drawings. For the purposes of illustrating the present disclosure, exemplary constructions of the disclosure are shown in the drawings. However, the present disclosure is not limited to a specific device, or a tool and instrumentalities disclosed herein. Moreover, those in the art will understand that the drawings are not to scale. Wherever possible, like elements have been indicated by identical numbers:

FIG. 1 illustrates an example representation of an environment related to at least some example embodiments of the present disclosure;

FIG. 2A illustrates an exploded view of a chastity device, in accordance with an embodiment of the present disclosure;

FIG. 2B illustrates a front view of the chastity device, in accordance with an embodiment of the present disclosure;

FIG. 2C illustrates a perspective view of the chastity device being secured to genitalia of a user, in accordance with an embodiment of the present disclosure;

FIG. 3A illustrates an exploded view of a housing of the chastity device, in accordance with an embodiment of the present disclosure;

FIG. 3B illustrates a perspective view of the housing depicting internal components of the housing, in accordance with an embodiment of the present disclosure;

FIG. 3C illustrates a bottom perspective view of the housing, in accordance with an embodiment of the present disclosure;

FIG. 4A illustrates a schematic representation of a set of actuators of the chastity device operated in a rest state on Hall Effect Sensors (HES), in accordance with an embodiment of the present disclosure;

FIG. 4B illustrates a schematic representation of the set of actuators of the chastity device operated in an excited state of the Hall Effect Sensors (HES), in accordance with an embodiment of the present disclosure;

FIG. 5 illustrates a flow diagram of a method for operating the chastity device, in accordance with an embodiment of the present disclosure; and

FIG. 6 illustrates a simplified block diagram representation of a control circuitry, in accordance with an embodiment of the present disclosure;

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features.

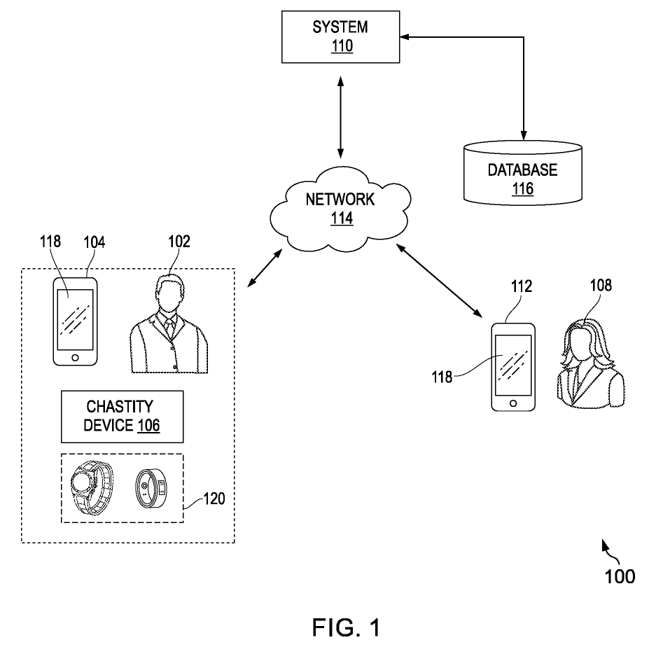

Various embodiments of the present disclosure are described hereinafter with reference to FIGS. 1 - 6.

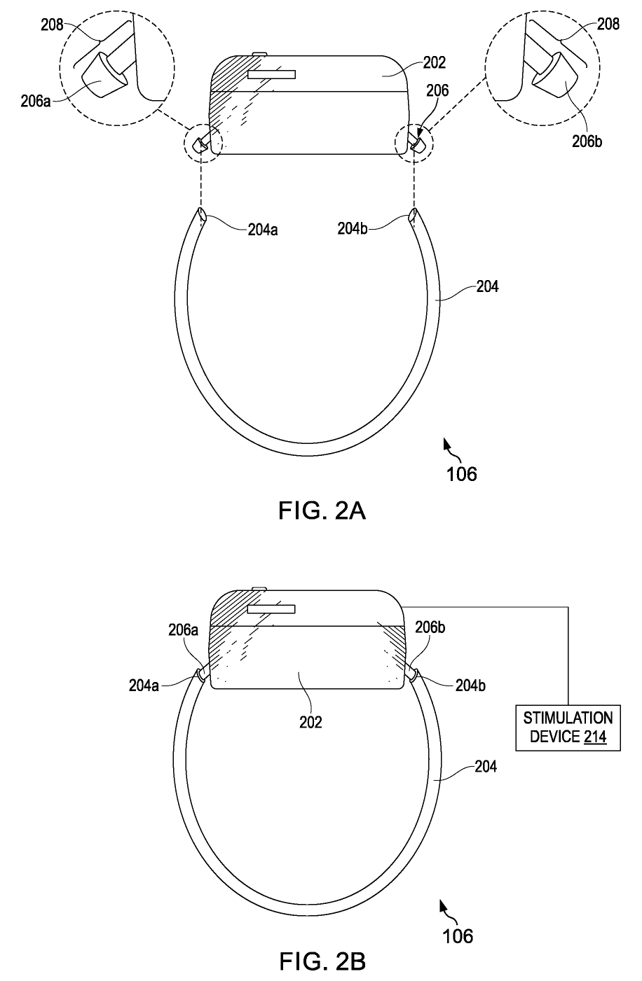

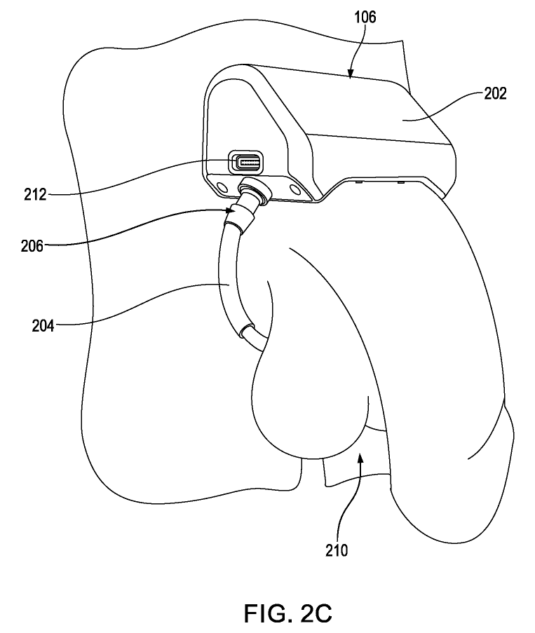

FIG. 1 illustrates an example representation of an environment 100 related to at least some example embodiments of the present disclosure. Although the environment 100 is presented in one arrangement, other arrangements are also possible where the parts of the environment 100 (or other parts) are arranged or interconnected differently. The environment 100 includes a user 102 associated with a user device 104 and at least one wearable device 120. The user device 104 may include at least a laptop computer, a phablet computer, a handheld personal computer, a Virtual Reality (VR) device, a netbook, a Web book, a tablet computing device, a smartphone, or other mobile computing devices. Further, the user 102 includes a chastity device 106. As shown, the user 102 is a male user, hence, the chastity device 106 corresponds to a male chastity device. The chastity device 106 is designed to be worn comfortably over a genital region (see, 210 of FIG. 2C) of the user 102 to prevent sexual activity or arousal, typically by restricting access to the genital region 210. The genital region 210 is interchangeably referred to as ‘the genitalia 210’. Further, the at least one wearable device 120 may include a smartwatch, a wearable fitness band, a smart ring, and the like. The at least one wearable device 120 may be communicably coupled to the user device 104 using short range communication protocols (e.g., Bluetooth, Zigbee, etc.). The at least one wearable device 120 may be configured to track, psychological metrics, sleeping hours, number of steps, number of calories burnt, and the like.

Further, the environment 100 includes a client user 108 associated with a user terminal 112 (exemplarily depicted to be ‘a mobile phone’). In an example, the client user 108 is depicted as a female user. The client user 108 may be an individual or an authority for the user 102 to operate and control the chastity device 106. In another example, the client user 108 may be a medical or clinical professional of the user 102, who is provided with the authority for controlling the chastity device 106 of the user 102 to treat issues related to sexual health, function, and well-being. Furthermore, the chastity device 106 may be communicably coupled to the user terminal 112 through one or more short-range communication protocols. Also, the chastity device 106 may be communicably coupled to the user device 104 using the one or more short-range communication protocols. Some non-limiting examples of the one or more short-range communication protocols include Bluetooth, Zigbee, Near Field Communication (NFC), Wireless Fidelity (Wi-Fi) Direct, and the like.

Various entities in the environment 100 may connect to a network 114 in accordance with various wired and wireless communication protocols, such as Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, Long Range (LoRa) Gateway Protocol or any combination thereof. In some instances, the network 114 may include a secure protocol (e.g., Hypertext Transfer Protocol (HTTP)), and/or any other protocol, or set of protocols. In an embodiment, the network 114 may include, without limitation, a local area network (LAN), a wide area network (WAN) (e.g., the Internet), a mobile network, a virtual network, and/or another suitable public and/or private network capable of supporting communication among two or more of the entities illustrated in FIG. 1, or any combination thereof.

The environment 100 further includes a system 110. In an embodiment, the system 110 may be embodied in at least one computing device in communication with the network 114. In an embodiment, the system 110 may be embodied in the user terminal 112. In another embodiment, the system 110 may be an individual entity located remotely and communicably coupled to the entities of FIG. 1 via the network 114. The system 110 may be specifically configured, via executable instructions, to perform one or more of the operations described herein. In general, the system 110 is configured to control the operation of the chastity device 110 based on the instructions from the client user 108, which will be explained further in detail.

Further, the system 110 may be configured to host and manage an application 118. The application 118 is a set of computer-executable codes configured to allow the client user 106 or the user 102 to control the operation of the chastity device 106. In one embodiment, the application 118 may be accessed as a web-based application on the user terminal 112 and the user device 104. In another embodiment, the user device 112 may access an instance of the application 118 from the system 110 for installation on the user terminal 112 and the user device 104 using application stores associated with operating systems such as Apple iOS®, Android™ OS, Google Chrome OS, Symbian OS®, Windows Mobile® OS, and the like. Further, the system 110 may be configured to trigger the control circuitry of the chastity device 106 to facilitate the connection between the chastity device 106 and the application 118 equipped in the user terminal 112 associated with the client user 108 using the one or more short communication protocols.

In an embodiment, the chastity device 106 operates in a first operating mode. In the first operating mode, the chastity device 106 may be self-operated based on sensory feedback of one or more sensors (not shown in FIG. 1) associated with the chastity device 106. In particular, the one or more sensors of the chastity device 106 may detect a set of parameters indicative of one or more events associated with at least the genitalia 210 and the chastity device 106. For example, the one or more events include a sexual arousal of the user and a tampering condition of the chastity device 106. Upon detecting the set of parameters related to the one or more events exceeding a set of threshold parameters, the chastity device 106 is configured to provide a stimulatory effect. The stimulatory effect may persist until the set of parameters indicative of the one or more events is determined to be within the set of threshold parameters. In other words, the stimulatory effect may persist until the one or more events cease.

In another embodiment, the chastity device 106 operates in a second operating mode. In the second operating mode, the chastity device 106 is communicably coupled to the user terminal 112 (or the application 118 equipped in the user terminal 112). This enables the client user 108 to monitor the set of parameters indicative of the one or more events of the user 102, configure settings, provide instructions to define the set of threshold parameters to trigger the stimulatory effect, alerts, and the like. Prior to providing access to the above services related to controlling the chastity device 106, the client user 108 may be required to complete a one-time registration process in the application 118 to avail the services associated with the application 118. In this scenario, the sensory feedback from the one or more sensors is rendered on the application 118. Herein, the operation of the chastity device 106 while the chastity device 106 is connected to the user terminal 112 corresponds to the second operating mode.

The number and arrangement of systems, devices, and/or networks shown in FIG. 1 are provided as an example. There may be other systems, devices, and/or networks; fewer systems, devices, and/or networks; different systems, devices, and/or networks, and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices.

FIG. 2A illustrates an exploded view of the chastity device 106, in accordance with an embodiment of the present disclosure. FIG. 2B illustrates a front view of the chastity device 106, in accordance with an embodiment of the present disclosure. FIG. 2C illustrates a perspective view of the chastity device 106 being secured to the genitalia 210 of the user 102, in accordance with an embodiment of the present disclosure.

The chastity device 106 includes a housing 202. The housing 202 is configured to enclose and support one or more components of the chastity device 106. The housing 202 is configured to be positioned over a portion of the genitalia 210 (for e.g., penile shaft) of the user 102. Further, the housing 202 may be formed from a variety of materials including, but not limited to, plastics, metals, composites, or any combination thereof. The housing 202 may be rigid or semi-flexible. The housing 202 may be configured as a modular structure. In other words, the housing 202 may be formed by removably coupling one or more parts to each other via conventional coupling means (e.g., fasteners, clips, adhesives, or snap-fit mechanisms). This configuration of the housing 202 allows easy access to the internal components housed in the housing 202. In an embodiment, the housing 202 may be configured with water-resistant elements and dust-proof elements as the housing 202 is in contact with the genitalia 210 of the user 102.

The chastity device 106 further includes a securing member 204. The securing member 204 is operatively coupled to the housing 202 while being secured to the genitalia 210 of the user 102 (as shown in FIG. 2C). In particular, the chastity device 106 includes a set of actuators 206. The set of actuators 206 includes an actuator 206a and an actuator 206b. The set of actuators 206 (or the actuators 206a and 206b) is disposed in the housing 202. Further, a portion (such as a portion 208) of the set of actuators 206 (or each of the actuators 206a and 206b) extends outwardly from the housing 202. The set of actuators 206 (or the portion 206a of the set of actuators 206) is operatively coupled to the securing member 204. Further, the securing member 204 may be designed to be a hollow structure. The hollow structure of the securing member 204 enables the securing member 204 to be secured to the portion 208 of the set of actuators 206. More specifically, the securing member 204 includes a first end 204a and a second end 204b. The first end 204a of the securing member 204 is secured to the portion 208 of the actuator 206a, and the second 204b of the securing member 204 is secured to the portion 208 of the actuator 206b, or vice versa. As explained above, the securing member 204 is secured over the genitalia 210 of the user 102. In particular, one end (e.g., the first end 204a) of the securing member 204 is initially secured to the portion 208 of one actuator (e.g., the actuator 206a). Thereafter, the securing member 204 is wound around the base of the penile shaft and positioned behind the scrotum area of the user 102 for securing another end (e.g., the second end 204b) of the securing member 204 to the portion 208 of another actuator (e.g., the actuator 206b). In this way, the securing member 204 is secured over (or encircles) the genitalia 210 of the user 102 while being operatively coupled to the housing 202 (as shown in FIG. 2C). Also, the securing member 204 serves as the anchor of the chastity device 106.

The securing member 204 may be formed from a rigid or semi-flexible material, such as stainless steel, polycarbonate, silicone, or other biocompatible substances. Further, the securing member 204 may be configured in various sizes to accommodate different anatomical dimensions of the genitalia 210. In some embodiments, the securing member 204 may be adjustable or segmented to allow for size customization and ease of application. The securing member 204 may be designed to be circular, oval, or ergonomically contoured to enhance the user’s 102 comfort and reduce pressure during use.

In one example, the securing member 204 may be an elastic band made from body-safe silicone elastomers or thermoplastic polyurethane (TPU) that stretches to accommodate size changes while maintaining gentle pressure around the penis and/or scrotum of the user 102. The elasticity must accommodate significant changes in penile circumference from a flaccid to an erect state without causing discomfort to the user 102. This ensures consistent contact of the securing member 204 with the skin for accurate sensor readings. In another example, the securing member 204 may be designed as a non-elastic cord with an adjustable mechanism. In this configuration, the securing member 204 may be constructed from soft, skin-friendly fabrics equipped with the adjustable mechanism, such as a clasp, buckle, or quick-release mechanism, to set the desired tension. In another example, the securing member 204 may be designed as a pouch-like structure. The pouch-like structure may be made from breathable and soft fabric.

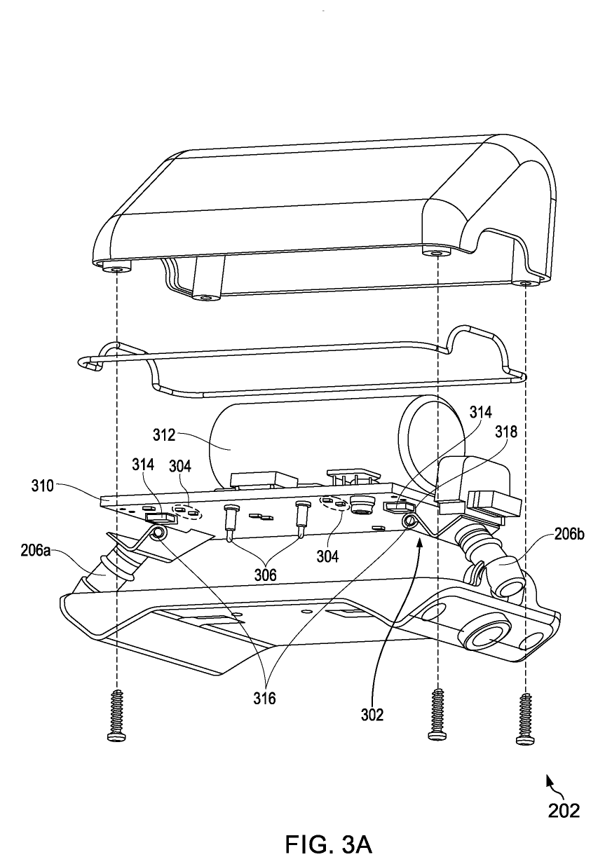

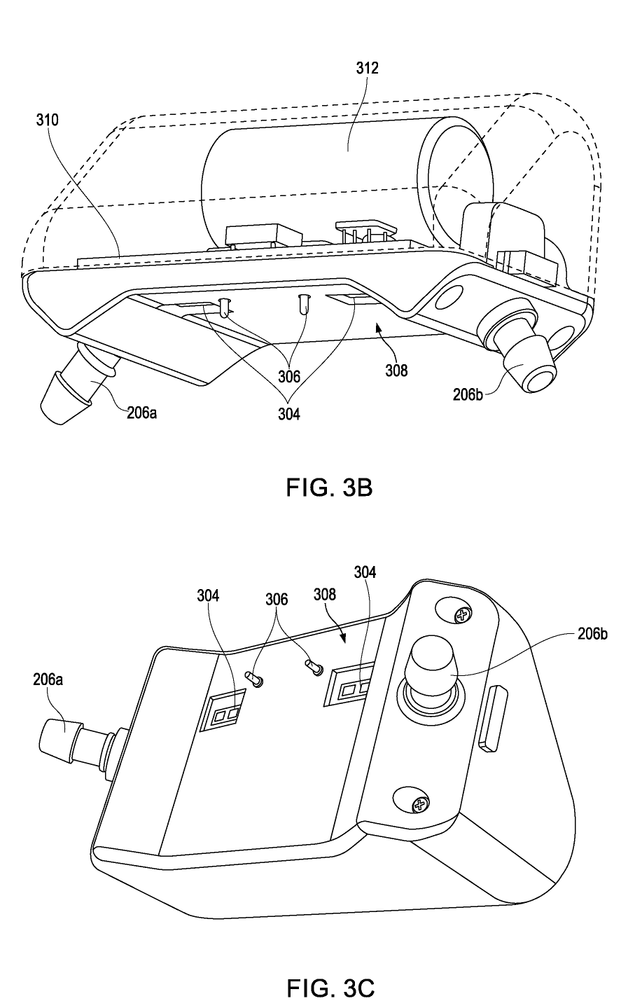

FIG. 3A illustrates an exploded view of the housing 202, in accordance with an embodiment of the present disclosure. FIG. 3B illustrates a perspective view of the housing 202, in accordance with an embodiment of the present disclosure. FIG. 3C illustrates a bottom perspective view of the housing 202, in accordance with an embodiment of the present disclosure.

The chastity device 106 further includes one or more sensors 302, at least one stimulation device (see, 214 of FIG. 2B), and control circuitry 310. The one or more sensors 302 are operatively coupled to the control circuitry 310. In one embodiment, the one or more sensors 302 (hereinafter interchangeably referred to as ‘the sensors 302’) may be disposed in the housing 202 (as shown in FIGS. 3A and 3B). The sensors 302 disposed in the housing 202 may be operatively coupled to the control circuitry (as shown in FIG. 3A). In another embodiment, the sensors 302 may be integrated into the control circuitry 310. For example, the sensors 302 may be mounted on the control circuitry 310 using soldering techniques to maintain a low profile and enhance comfort when worn around sensitive areas. This configuration ensures durability and consistent placement of the sensors 302 on the control circuitry 310. In another embodiment, the sensors 302 may be a separate entity and are communicably coupled to the control circuitry 310 and the securing member 204. For example, the sensors 302 are attached externally at specific points on the securing member 204. This configuration allows easy maintenance and replacement, and facilitates modularity. In another embodiment, the sensors 302 may be attached or detached from the chastity device 106 (or the securing member 204) based on the user’s preferences. In another embodiment, the sensors 302 may be distributed along the control circuitry 310 to increase accuracy and provide comprehensive data collection from both the penile and scrotal regions of the user 102.

The control circuitry 310 is disposed in the housing 202 (as shown in FIGS. 3A and 3B).The control circuitry 310 is communicably coupled to the sensors 302, the at least one stimulation device 214, and the securing member 204. The control circuitry 310 is configured to control and operate the chastity device 106.

The chastity device 106 further includes a power source (see, 312 of FIG. 3A). The power source 312 may be disposed in the housing 202 (as shown in FIGS. 3A and 3B). The power source 312 provides power supply to at least the control circuitry 310, the sensors 302, the at least one stimulation device 214, and any other electronic components of the chastity device 106. The power source 312 may provide one of an alternating current output or a direct current output. In an embodiment, the power source 312 includes a direct current power source, such as a rechargeable battery (for example, a lithium-ion battery), operable to provide the required electrical power for the operation of the chastity device 106. Further, the power source 312 may include electrical and/or electronic components or circuits for enabling the use of wired or wireless charging. Alternatively, the power source 312 may include electrical and/or electronic components or circuits for enabling the use of alternating current to provide the required electrical power for the operation of the chastity device 106. Further, the chastity device 106 may include a charging port (see, 212 of FIG. 2C) to plug an electric line for receiving electric power for charging the power source 312.

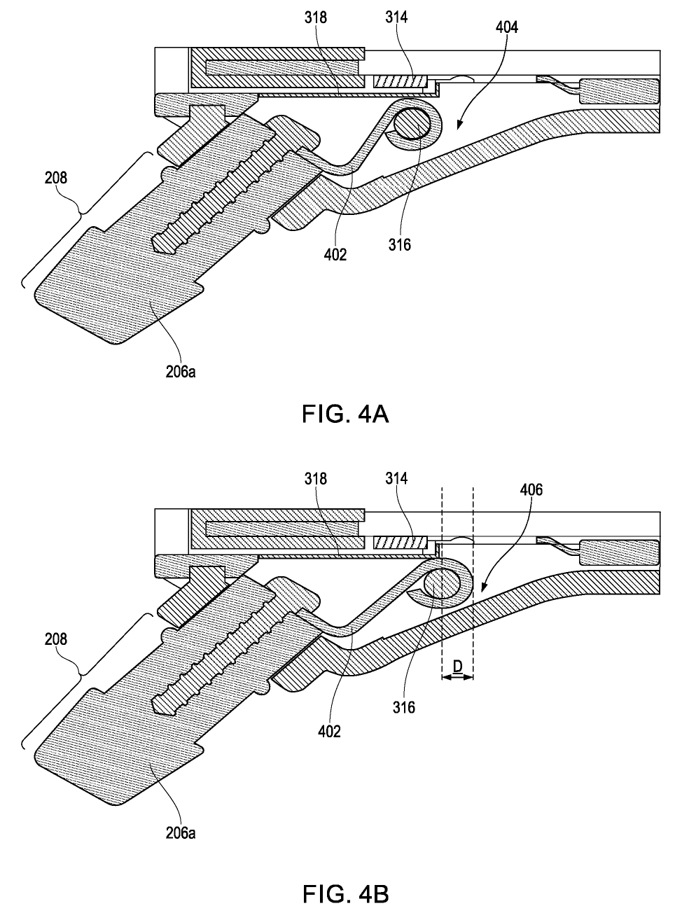

Referring to FIGS. 4A and 4B, the sensors 302 may include a Hall Effect Sensor (HES) 314. Further, the Hall Effect Sensor (HES) 314 is operatively coupled to the control circuitry 310. Further, the set of actuators 206 (or the actuators 206a and 206b) is coupled with a contacting member (see, 402 of FIGS. 4A and 4B). The contacting member 402 may include, but is not limited to, a sliding element, a retracting element, a tether element, a connector element, a clamp member, a deflecting member, and the like. The contacting member 402 is coupled with at least one magnetic member 316. The at least one magnetic member 316 is mounted to the contacting member 402 in such a way that the Hall Effect Sensor (HES) 314 is in the vicinity of the at least one magnetic member 316. The set of actuators 206 causes a displacement of the at least one magnetic member 316 coupled to the contacting member 402 based on the set of structural adaptations of the securing member 204 due to anatomical changes associated with the genitalia of the user 102. Additionally, the at least one magnetic member 316 and the Hall Effect Sensor (HES) 314 are separated by an enclosure 318. The enclosure 318 may be made of nonmagnetic materials such as plastic, rubber, and the like. The enclosure 318 allows the magnetic coupling of the at least one magnetic member 316 to the Hall Effect sensor (HES) 314 without direct contact. In this scenario, the Hall Effect sensor (HES) 314 is configured to detect a magnetic flux due to the displacement of the contacting member 402. The control circuitry 310 receives sensory data including the magnetic flux from the Hall Effect Sensor (HES) 314. The control circuitry 310 determines the set of parameters indicative of the one or more events based on the sensory data. Additionally, the sensory data may include head-on motion, lateral slide motion, rotational motion, and the like of the at least one magnetic member 316 with respect to the Hall Effect Sensor (HES) 314. Further, the control circuitry 310 determines the one or more events based at least on a change in the magnetic flux detected by the Hall Effect Sensor (HES) 314.

It is to be noted that the set of actuators 206 is configured to vary the position of the at least one magnetic member 316 through the contacting member 402. The position of the contacting member 402 associated with the at least one magnetic member 316 displaces with respect to the Hall Effect Sensor (HES) 314. As the at least one magnetic member 316 is displaced, the Hall Effect Sensor (HES) 314 detects a variation in the magnetic flux. The control circuitry 310 determines the set of parameters based on the variation in the magnetic flux. The set of parameters is indicative of the one or more events, including at least a sexual arousal of the user 102 and a tampering condition associated with the chastity device 106. The set of parameters includes at least one of displacement, tension level, strain, force, and the like associated with the securing member 204 of the chastity device 106 based on the sexual arousal of the genitalia 210 of the user 102.For example, the anatomical changes associated with the genitalia 210 of the user 102 may include sexual arousal of the penile shaft from a flaccid state to an erect state. In particular, the set of actuators 206 is in a rest state (see, 404 of FIG. 4A) while there are no anatomical changes in the genitalia 210 of the user 102 i.e., the penile shaft of the user 102 may be in the flaccid state. In this scenario, there is no change in magnetic flux detected by the sensors 302 (such as the HES 314) as the change in the position of the contacting member 402 associated with the at least one magnetic member 316 is caused by the set of actuators 206, which is negligible.

Further, the set of actuators 206 is operated in an excited state (see, 406 of FIG. 4B) due to the structural adaptations of the securing member 204 as a result of the anatomical changes associated with the genitalia 210 of the user 102. In other words, the set of actuators 206 changes the position of the contacting member 402 associated with the at least one magnetic member 316 due to the structural adaptations of the securing member 204 due to the anatomical changes associated with the genitalia 210 of the user 102. For illustration purposes, the excited state 406 is depicted as the change in the position of the contacting member 402 associated with the at least one magnetic member 316 due to the anatomical changes associated with genitalia 210 operating the set of actuators 206 in the excited state.

Further, the control circuitry 310 is configured to determine reference calibration data for each user associated with the chastity device 106 through the combined operation of the securing member 204 and the Hall Effect Sensor (HES) 314 while the genitalia 210 being in at least the flaccid state and the substantially erect state. The reference calibration data includes a reference magnetic flux and a corresponding tension level. In one embodiment, the reference calibration data of the chastity device 106 may be guided by the client user 108 once the chastity device 106 is communicably coupled to the user terminal 112 of the client user 108. During the determination of the reference calibration data, the control circuitry 310 detects the set of parameters indicative of the sexual arousal of the genitalia 210 of the user 102 through the combined operation of the securing member 204 and the one or more sensors 302. The set of parameters indicative of the sexual arousal may include a tension level around the genitalia 210 and the reference magnetic flux while the genitalia 210 is in at least a flaccid state and a substantially erect state. The reference calibration data is stored in a memory (see, 604 in FIG. 6) associated with the chastity device 106 identifying the one or more events associated with the user 102 using the chastity device 106.

Furthermore, the control circuitry 310 is configured to determine whether the set of parameters indicative of the one or more events is within the set of threshold parameters. If the control circuitry 310 determines that the set of parameters indicative of the one or more events exceeds the set of threshold parameters, the control circuitry 310 generates a control signal. The control signal is transmitted to the at least one stimulation device 214. For illustration purposes, the at least one stimulation device 214 is depicted to be as an external entity and is connected to the chastity device 106. However, it is to be noted that the at least one stimulation device 214 may be disposed in the housing 202, or at any other locations as per the design feasibility and requirements. The control circuitry 310 transmits the control signal to the at least one stimulation device 214. The control signal operates the at least one stimulation device 214 to provide the stimulatory effect until the set of parameters indicative of the one or more events is within the set of threshold parameters.

In one scenario, the at least one stimulation device 214 may be configured to apply an electrical stimulation to the genitalia 210 of the user 102 in response to receipt of the control signal. Herein, the electrical stimulation applied to the genitalia 210 of the user 102 corresponds to the stimulatory effect. The electrical stimulation may be provided for a specific duration (e.g., every 5 or 10 seconds) until the set of parameters associated with the one or more events is within the set of threshold parameters. In other words, the at least one stimulation device 214 provides continuous electrical shocks in response to the control signal. The electrical shocks persist until the detected events cease, such as a reduction in tension level within the set of threshold parameters or the cessation of the tampering condition.

Additionally, the sensors 302 may include other types of sensors for detecting the set of parameters, such as the set of parameters related to movement associated with the sexual arousal of the genitalia 210 of the user 102.

Thus, it is to be noted that the sensors 302 associated with the chastity device 106 are configured to the set of parameters indicative of the one or more events such as the biometric data, the tension level associated with the securing member 204, and the at least one parameter related to movement associated with the sexual arousal of the genitalia 210 of the user 102. Thereafter, the sensors 302 may generate sensory data based on the detected set of parameters associated with the one or more events and transmit the sensory data to the control circuitry 310 for further analysis.

The control circuitry 310 generates a data representation of the sensory data indicative of the one or more events. The digital data representation may be generated by at least one of a lookup table (LUT), a polynomial and/or piecewise mapping, a scaling and offset compensation, a temperature and/or drift compensation, and the like. The digital data representation may include the digital form of the displacement, the tension level, the arousal state of the genitalia 210, and the like. In one embodiment, the chastity device 106 is configured to operate in the second operating mode. The control circuitry 310 facilitates a connection between the chastity device 106 and the application 118 equipped in the user terminal 112 associated with the client user 108 using one or more short communication protocols. Further, the control circuitry 310 transmits the digital data representation including the set of parameters indicative of one or more events associated with at least the genitalia 210 and the chastity device 106 to the user terminal 112 of the client user 108 to provide the control signals to operate the at least one stimulation device 214 associated with the chastity device 106.

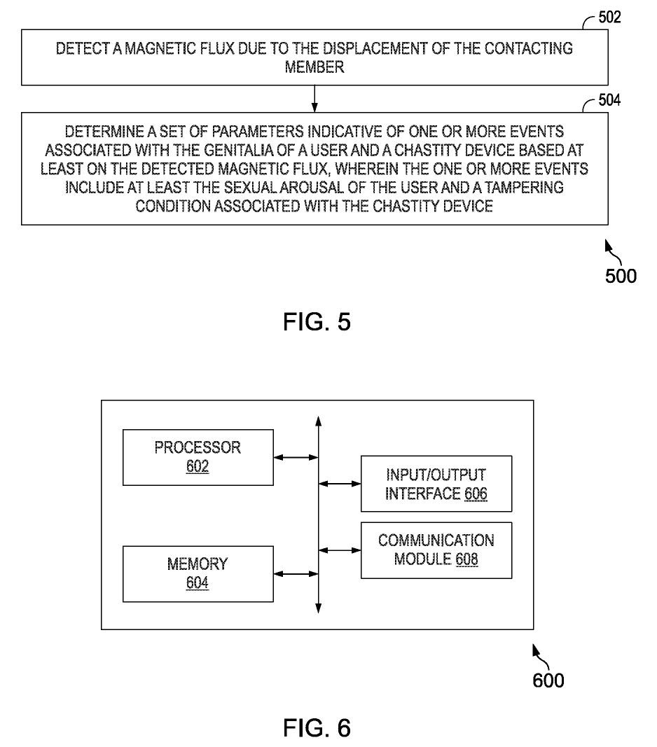

FIG. 5 illustrates a flow diagram of a method 500 for operating the chastity device 106, in accordance with an embodiment of the present disclosure. The method 500 depicted in the flow diagram may be executed by, for example, the control circuitry 310. Operations of the flow diagram of the method 500, and combinations of the operations in the flow diagram of the method 500, may be implemented by, for example, hardware, firmware, a processor, circuitry, and/or a different device associated with the execution of software that includes one or more computer program instructions. The method 500 starts at operation 502.

At operation 502, the method 500 includes detecting, by the one or more sensors 302, the magnetic flux due to the displacement of the contacting member 402.

At operation 504, the method 500 includes determining, by the control circuitry 310, the set of parameters indicative of one or more events associated with the genitalia of the user 102 and the chastity device 106 based at least on the detected magnetic flux. The one or more events include at least the sexual arousal of the user 102 and the tampering condition associated with the chastity device 106.

FIG. 6 illustrates a simplified block diagram representation of a control circuitry 600, in accordance with an embodiment of the present disclosure. The control circuitry 600 is an example of the control circuitry 310. The control circuitry 600 includes at least one processor, such as a processor 602 and a memory 604. It is noted that although the control circuitry 600 is depicted to include only one processor, the control circuitry 600 may include more processors therein. In an embodiment, the memory 604 is capable of storing machine-executable instructions. Further, the processor 602 is capable of executing the machine-executable instructions to perform one or more operations described herein. In an embodiment, the processor 602 may be embodied as a multi-core processor, a single-core processor, or a combination of one or more multi-core processors and one or more single-core processors. For example, the processor 602 may be embodied as one or more of various processing devices, such as a coprocessor, a microprocessor, a controller, a Digital Signal Processor (DSP), a processing circuitry with or without an accompanying DSP, or various other processing devices including integrated circuits such as, for example, an Application Specific Integrated Circuit (ASIC), a Field Programmable Gate Array (FPGA), a microcontroller unit (MCU), a hardware accelerator, a special-purpose computer chip, or the like. In an embodiment, the processor 602 may be configured to execute hard-coded functionality. In an embodiment, the processor 602 is embodied as an executor of software instructions, wherein the instructions may specifically configure the processor 602 to perform the algorithms and/or operations described herein when the instructions are executed.

The memory 604 may be embodied as one or more volatile memory devices, one or more non-volatile memory devices, and/or a combination of one or more volatile memory devices and non-volatile memory devices. For example, the memory 604 may be embodied as semiconductor memories (such as mask (ROM), programmable ROM (PROM, Erasable PROM (EPROM), flash memory, Random Access Memory (RAM), etc.), magnetic storage devices (such as hard disk drives, floppy disks, magnetic tapes, etc.), optical magnetic storage devices (e.g., magneto-optical disks), Compact Disc Read Only Memory (CD-ROM), Compact Disc Recordable (CD-R), Compact Disc Rewritable (CD-R/W), Digital Versatile Disc (DVD) and BLU-RAY® Disc (BD).

The control circuitry 600 further includes an Input/Output (I/O) module 606 (hereinafter referred to as an ‘I/O module 1006’) and at least one communication module such as a communication module 608. In an embodiment, the I/O module 606 may include mechanisms configured to receive inputs (or data from the sensors 302) and provide outputs such as the control signal to trigger the stimulation device 214 or provide the stimulatory effect in response to the control signal from the processor 602.

In an embodiment, the processor 602 may include I/O circuitry configured to control at least some functions of one or more elements of the I/O module 606, such as, for example, a speaker, a microphone, a display, and/or the like. The processor 602 and/or the I/O circuitry may be configured to control one or more functions of the one or more elements of the I/O module 606 through computer program instructions, for example, software and/or firmware, stored on a memory, for example, the memory 604, and/or the like, accessible to the processor 602.

The communication module 608 may include communication circuitry such as, for example, a transceiver circuitry including an antenna and other communication media interfaces to connect to a wired and/or wireless communication protocol. The communication circuitry may, in at least some example embodiments, enable the transmission of data signals and/or reception of signals from other network entities, such as the sensors 302, the user device 104, the user terminal 112, the at least one wearable device 120, or other entities of FIG. 1.

In an embodiment, the processor 602 receives the data from at least the sensors 302 and the at least one wearable device 120 via a communication module (such as the communication module 608). The processor 1002 is configured to operate the chastity device 106 in the first operating mode and the second operating mode based on the connectivity between the chastity device 106 and the user terminal 112. Further, the processor 602 may be configured to detect whether the set of parameters indicative of the one or more events is within the set of threshold parameters. The processor 602 may be further configured to operate the at least one stimulation device 214 if the set of parameters exceeds the set of threshold parameters. The set of threshold parameters may be stored in the memory 404. Furthermore, the processor 1002 may be configured to calibrate the chastity device 106. The processor 602 may transmit the sensory data to the user terminal 118. The one or more operations performed by the control circuitry 310 are explained above, therefore they are not reiterated herein for the sake of brevity.

Various embodiments of the disclosure, as discussed above, may be practiced with steps and/or operations in a different order, and/or with hardware elements in configurations, which are different than those which are disclosed. Therefore, although the disclosure has been described based on these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A chastity device, comprising:
a housing;
a securing member operatively coupled to the housing while being secured to genitalia of a user;
at least one magnetic member coupled to a contacting member and disposed in the housing;
one or more sensors configured to detect a magnetic flux due to a displacement of the contacting member; and
control circuitry disposed in the housing, the control circuitry communicably coupled to the one or more sensors, wherein the control circuitry is configured to determine a set of parameters indicative of one or more events associated with the genitalia of the user and the chastity device based at least on the detected magnetic flux, wherein the one or more events comprise at least a sexual arousal of the user and a tampering condition associated with the chastity device.

2. The chastity device as claimed in claim 1, further comprising an enclosure disposed between the at least one magnetic member and the one or more sensors.

3. The chastity device as claimed in claim 1, wherein the set of parameters indicative of the one or more events comprises at least the displacement, tension level, strain, and force associated with the securing member based on the sexual arousal of the genitalia of the user.

4. The chastity device as claimed in claim 3, wherein the control circuitry is further configured at least, in part to:
generate a data representation of the sensory data indicative of the one or more events;
facilitate a connection between the chastity device and an application equipped in a user terminal associated with a client user using one or more short communication protocols; and
transmit the digital data representation comprising the set of parameters indicative of the one or more events associated with at least the genitalia and the chastity device to the user terminal of the client user.

5. The chastity device as claimed in claim 1, further comprising a set of actuators disposed in the housing, and a portion of the set of actuators extends outwardly from the housing, the set of actuators operatively coupled to the securing member being secured over the genitalia of the user, and the one or more sensors.

6. The chastity device as claimed in claim 5, wherein the set of actuators is configured to cause the displacement of the at least one magnetic member coupled to the contacting member based on the structural adaptations of the securing member due to anatomical changes associated with the genitalia of the user.

7. The chastity device as claimed in claim 6, wherein the set of actuators is operated in an excited state from a rest state due to the structural adaptations of the securing member corresponding to the anatomical changes associated with the genitalia of the user, thereby causing the displacement of the at least one magnetic member magnetically coupled to the one or more sensors.

8. The chastity device as claimed in claim 1, further comprising:
at least one stimulation device communicably coupled to the securing member and the control circuitry; and
wherein control circuitry is further configured to:
determine reference calibration data associated with a set of threshold parameters for each user associated with the chastity device through the combined operation of the securing member and the one or more sensors while the genitalia being in at least the flaccid state and the substantially erect state, the reference calibration data comprising a reference magnetic flux and a corresponding tension level, and wherein the reference calibration data is stored in a memory associated with the chastity device for identifying the one or more events associated with the user using the chastity device; and
operate the at least one stimulation device to provide a stimulatory effect based on the set of parameters being detected by the one or more sensors of the chastity device, wherein the at least one stimulation device comprises an electrical stimulation applied to the genitalia.

9. The chastity device as claimed in claim 8, wherein the control circuitry is further configured at least, in part to:
receive sensory data comprising the set of parameters indicative of the one or more events associated with at least the genitalia and the chastity device by the one or more sensors,
determine whether the set of parameters indicative of the one or more events is within the set of threshold parameters,
generate a control signal based on determining the set of parameters indicative of the one or more events exceeding the set of threshold parameters, and
transmit the control signal to at least one stimulation device, wherein the control signal operates the at least one stimulation device to provide a stimulatory effect until the set of parameters indicative of the one or more events is within the set of threshold parameters.

10. A method, comprising:
detecting, by one or more sensors, a magnetic flux due to a displacement of a contacting member; and
determining, by control circuitry, a set of parameters indicative of one or more events associated with the genitalia of the user and the chastity device based at least on the detected magnetic flux, wherein the one or more events comprise at least a sexual arousal of the user and a tampering condition associated with the chastity device.

11. The method as claimed in claim 10, wherein the set of parameters indicative of the one or more events comprises at least the displacement, tension level, strain, and force associated with the securing member based on the sexual arousal of the genitalia of the user.

12. The method as claimed in claim 11, further comprising:
generating, by the control circuitry, a data representation of the sensory data indicative of the one or more events;
facilitating, by the control circuitry, a connection between the chastity device and an application equipped in a user terminal associated with a client user using one or more short communication protocols; and
transmitting, by the control circuitry, the digital data representation comprising the set of parameters indicative of the one or more events associated with at least the genitalia and the chastity device to the user terminal of the client user.

13. The method as claimed in claim 10, further comprising:
determining, by the control circuitry, reference calibration data associated with a set of threshold parameters for each user associated with the chastity device through the combined operation of the securing member and the one or more sensors while the genitalia being in at least the flaccid state and the substantially erect state, the reference calibration data comprising a reference magnetic flux and a corresponding tension level, and wherein the reference calibration data is stored in a memory associated with the chastity device for identifying the one or more events associated with the user using the chastity device; and
operating, by the control circuitry, the at least one stimulation device to provide a stimulatory effect based on the set of parameters being detected by the one or more sensors of the chastity device, wherein the at least one stimulation device comprises an electrical stimulation applied to the genitalia.

14. The method as claimed in claim 13, further comprising:
receiving, by the control circuitry, sensory data comprising the set of parameters indicative of the one or more events associated with at least the genitalia and the chastity device by the one or more sensors,
determining, by the control circuitry, whether the set of parameters indicative of the one or more events is within the set of threshold parameters,
generating by the control circuitry, a control signal based on determining the set of parameters indicative of the one or more events exceeding the set of threshold parameters, and
transmitting, by the control circuitry, the control signal to at least one stimulation device, wherein the control signal operates the at least one stimulation device to provide a stimulatory effect until the set of parameters indicative of the one or more events is within the set of threshold parameters.

15. A chastity device, comprising:
a housing;
a securing member operatively coupled to the housing while being secured to genitalia of a user;
at least one magnetic member coupled to a contacting member and disposed in the housing;
one or more sensors configured to detect a magnetic flux due to a displacement of the contacting member;
an enclosure disposed between the at least one magnetic member and the one or more sensors; and
control circuitry disposed in the housing, the control circuitry communicably coupled to the one or more sensors, wherein the control circuitry is configured to determine a set of parameters indicative of one or more events associated with the genitalia of the user and the chastity device based at least on the detected magnetic flux, wherein the one or more events comprise at least a sexual arousal of the user and a tampering condition associated with the chastity device.

16. The chastity device as claimed in claim 15, further comprising a set of actuators disposed in the housing, and a portion of the set of actuators extends outwardly from the housing, the set of actuators operatively coupled to the securing member being secured over the genitalia of the user, and the one or more sensors.

17. The chastity device as claimed in claim 16, wherein the set of actuators is configured to cause the displacement of the at least one magnetic member coupled to the contacting member based on the structural adaptations of the securing member due to anatomical changes associated with the genitalia of the user.

18. The chastity device as claimed in claim 17, wherein the set of actuators is operated in an excited state from a rest state due to the structural adaptations of the securing member corresponding to the anatomical changes associated with the genitalia of the user, thereby causing the displacement of the at least one magnetic member magnetically coupled to the one or more sensors.

19. The chastity device as claimed in claim 15, further comprising:
at least one stimulation device communicably coupled to the securing member and the control circuitry; and
wherein control circuitry is further configured to:
determine reference calibration data associated with a set of threshold parameters for each user associated with the chastity device through the combined operation of the securing member and the one or more sensors while the genitalia being in at least the flaccid state and the substantially erect state, the reference calibration data comprising a reference magnetic flux and a corresponding tension level, and wherein the reference calibration data is stored in a memory associated with the chastity device for identifying the one or more events associated with the user using the chastity device; and
operate the at least one stimulation device to provide a stimulatory effect based on the set of parameters being detected by the one or more sensors of the chastity device, wherein the at least one stimulation device comprises an electrical stimulation applied to the genitalia.

20. The chastity device as claimed in claim 19, wherein the control circuitry is further configured at least, in part to:
receive sensory data comprising the set of parameters indicative of the one or more events associated with at least the genitalia and the chastity device by the one or more sensors,
determine whether the set of parameters indicative of the one or more events is within the set of threshold parameters,
generate a control signal based on determining the set of parameters indicative of the one or more events exceeding the set of threshold parameters, and
transmit the control signal to at least one stimulation device, wherein the control signal operates the at least one stimulation device to provide a stimulatory effect until the set of parameters indicative of the one or more events is within the set of threshold parameters.
